(19) United States
(12) Patent Application Publication
(10) Pub. No.: US 2014/0113321 A1
(43) Pub. Date: Apr. 24, 2014

(54) IDENTIFICATION OF CELLULOLYTIC MICROORGANISM CONTAMINATION IN FOOD AND OTHER MATERIALS

(71) Applicants: Ayyappan Nair, Trivandrum (IN); Ramya Govindaswamy, Bangalore (IN); Sunilkumar Sukumaran, Bangalore (IN); Madhuri Subbiah, Bangalore (IN); Kannan Thanukrishnan, Bangalore (IN); Ganesh Sambasivam, Bangalore (IN); Ravindra Chandrappa, Bangalore (IN); Ajay Bharadwaj, Bangalore (IN)

(72) Inventors: Ayyappan Nair, Trivandrum (IN); Ramya Govindaswamy, Bangalore (IN); Sunilkumar Sukumaran, Bangalore (IN); Madhuri Subbiah, Bangalore (IN); Kannan Thanukrishnan, Bangalore (IN); Ganesh Sambasivam, Bangalore (IN); Ravindra Chandrappa, Bangalore (IN); Ajay Bharadwaj, Bangalore (IN)

(73) Assignee: EMPIRE TECHNOLOGY DEVELOPMENT LLC, Wilmington, DE (US)

(21) Appl. No.: 14/125,222

(22) PCT Filed: Aug. 7, 2012
(86) PCT No.: PCT/IB12/54023

§ 371 Date: Dec. 10, 2013

(30) Foreign Application Priority Data
Mar. 21, 2012 (IN) 1040-CHE/2012

(51) Int. Cl. C12Q 1/34

(52) U.S. Cl. CPC C12Q 1/34
USPC 435/18

(57) ABSTRACT

Compositions, kits, and methods for detecting cellulolytic microorganisms in a sample are disclosed herein. In some embodiments cellulolytic microorganisms are detected by detecting the presence of a secreted enzyme, such as but not limited to cellobiohydrolase.

### BACKGROUND

Bacteria are the causative factor in many diseases of humans, animals and plants, and are commonly transmitted by carriers such as water, beverages, food and various organisms. Protection from deleterious microbial contaminants is a global issue. Each year millions of people throughout the world become ill and thousands die from contaminated food and water.

It is estimated that the industrial market for detection of microbial contaminants was approximately 600 million tests in 1997, amounting to a value of approximately USD 2.5 billion. Of the tests performed annually, the food segment is by far the largest segment, with approximately 310 million tests (53%), followed by the pharmaceutical segment with approximately 200 million tests (32%), the beverage segment with approximately 60 million tests (10%) and finally the environmental segment with approximately 30 million tests (5%). The majority of today's testing is performed with slow traditional methods (giving results in 2-3 days), which are laborious and expensive to use. These methods typically use agar plates or standard pour plates (plastic dishes with a nutrient medium), enhancing bacterial growth so that they multiply and their presence can be identified visually as colonies and counted. Accordingly, there is a need for more effective measurements that lead to more rapid and easy to use methods from both the public health and economical perspectives.

### SUMMARY

Some embodiments described herein are directed to methods of detecting cellulolytic microorganisms in a sample. In some embodiments, the method comprises contacting the sample with a cellobiohydrolase substrate and detecting cellobiohydrolase activity, in which detection of cellobiohydrolase activity indicates a cellulolytic microorganism is present in the sample.

Some embodiments provide kits for detection of cellulolytic microorganisms in a sample. In some embodiments, the kit comprises a cellobiohydrolase substrate comprising an indicator moiety; instructions for performing a method of detecting bacteria in a sample, one or more of a positive control; one or more of a negative control, or any combination thereof. In some embodiments, the kit provides instructions and/or reagents for a method of detecting cellulolytic microorganisms in a sample. In some embodiments, the method comprises contacting the sample with a cellobiohydrolase substrate, and detecting cellobiohydrolase activity, in which detection of cellobiohydrolase activity indicates a cellulolytic microorganism is present in the sample.

Some embodiments provide compositions comprising a sample, a cellobiohydrolase, a cellobiohydrolase substrate comprising an indicator moiety, or any combination thereof.

The foregoing summary is illustrative only and is not intended to be in any way limiting. In addition to the illustrative aspects, embodiments, and features described above, further aspects, embodiments, and features will become apparent by reference to the drawing and the following detailed description.

### BRIEF DESCRIPTION OF THE FIGURES

FIG. 1 is a schematic representation of substrate hydrolysis.

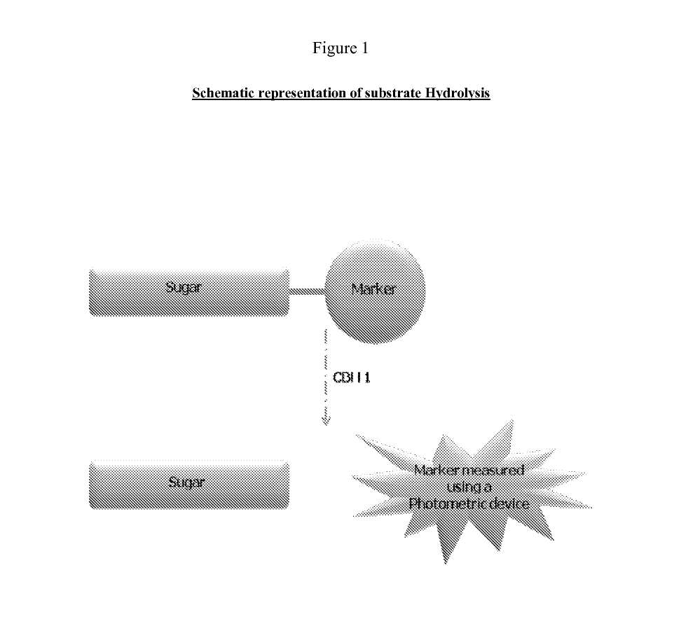

FIG. 2 is depicts the mechanism of cellulolysis by CBH I on a contaminated food substance.

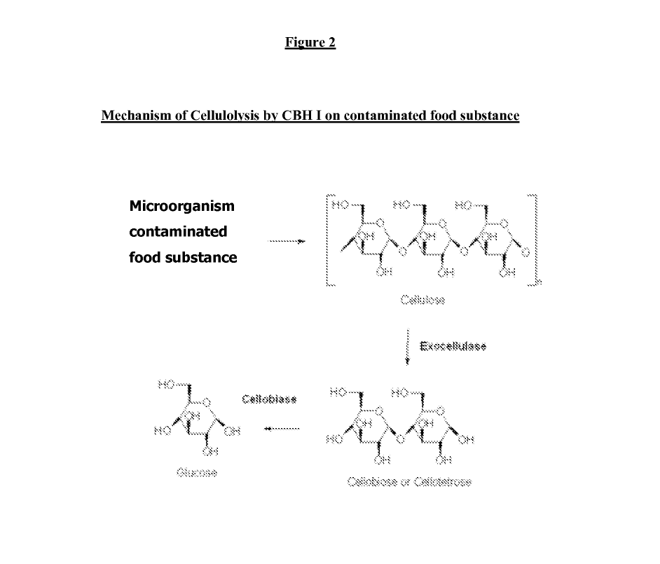

### DETAILED DESCRIPTION

In the following detailed description, reference is made to the accompanying drawing, which forms a part hereof. In the drawing, similar symbols typically identify similar components, unless context dictates otherwise. The illustrative embodiments described in the detailed description, drawing, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figure, can be arranged, substituted, combined, separated, and designed in a wide variety of different configurations, all of which are explicitly contemplated herein.

Cellulase, as used herein, refers to a class of enzymes that catalyze the hydrolysis of cellulose. The enzymes can be derived from or produced by, for example, but not limited to, fungi, bacteria, and protozoans. The hydrolysis of cellulose can result in the formation of glucose, cellobiose, cellooligosaccharides, and the like. Examples of cellulases include, but are not limited to, endo-cellulases, exo-cellulases, which can also be referred to as cellobiohydrolases (“CBH”), and beta-glucosidases, which can also be referred to as [beta]-D-glucoside glucohydrolase (“BG”). Endocellulases act mainly on the amorphous parts of the cellulose fiber, whereas cellobiohydrolases are able to also degrade crystalline cellulose.

As used herein, the term “Cellobiohydrolase” (CBH) refers to an exo-cellulase that hydrolyzes the β-1,4-linkages of a cellulose chain from its reducing end liberating β-cellobiose as the main product. For example, a cellobiohydrolase (1,4-b-D-glucan cellobiohydrolase, EC3.2.1.91) can release cellobiose units from the chain ends and degrade cellulose (e.g. crystalline cellulose) in a progressive manner. An example of a cellobiohydrolase includes, but is not limited to, CBH-1.

Microorganisms, such as, but not limited to, fungi and bacteria, rely on secreted hydrolytic enzymes, such as cellobiohydrolases, for the breakdown of extracellular polysaccharides into carbon sources. The breakdown products are readily taken up by the microbe and metabolized. The enzymes that are secreted by the microorganisms, including but not limited to, cellobiohydrolases, can then be used as a marker for microorganism contamination or as a marker for the presence of a microorganism in a sample. The enzymes can be used, for example, to catalyze a reaction that is used as indicator of a microorganism being present in a sample. The microorganism can be, for example, a bacteria or fungi. Examples of microorganisms are described herein and the methods described herein can be used to detect microorganisms in general and the ones specifically described. The methods can be used, for example, to detect cellulolytic microorganisms. A “cellulolytic microorganism” is a microorganism that secretes a cellobiohydrolase. Examples of cellulolytic microorganisms include bacteria (e.g. aerobic and anaerobic) and fungus (e.g. aerobic and anaerobic). Non-limiting examples of cellulolytic microorganisms are described herein and known to one of skill in the art. In some embodiments, the cellobiohydrolase is CBH-1.

Some embodiments provide methods of detecting the presence of microorganisms in a sample by detecting a microorganism's enzymatic activity. In some embodiments, the microorganism enzyme is a secreted microorganism enzyme. In some embodiments, the secreted enzyme is a cellobiohydrolase. Examples of cellobiohydrolase include, but are not limited to, CBH-1. The detection of the enzyme and its corresponding activity can be used for example to assay or monitor food contamination. The detection of the enzyme can also be used in a correlative manner to detect the presence of microorganisms in a sample.

Examples of samples that could harbor a microorganism (e.g. bacteria or fungi) and a secreted enzyme include, but are not limited to, food, soil, water, waste, and the like. In some embodiments, the food is milk, meat (e.g. beef, poultry, pork), produce (e.g. fruit, leafy green vegetables, and the like), grains, drinking water, and the like. Any sample can be analyzed to detect the presence or absence of the bacteria and/or the secreted enzyme, such as, but not limited to, cellobiohydrolase. Additionally, the sample can be generated by swabbing an item and culturing the swab to generate the sample. The sample can then be analyzed or processed according to the methods described herein to detect the presence or absence of bacteria in the sample. For example, a solid or semi-solid surface, such as, but not limited to, a door knob, a sink, a counter, a table, could be swabbed. The swab could then be cultured to create a sample and the sample could be processed or analyzed according to the methods described herein to detect the presence or absence of microorganisms. In some embodiments, the sample can be processed prior to determining whether microorganisms are present. In some embodiments, the processing can be purifying the sample to remove contamination. In some embodiments, the processing comprises culturing the sample. The culturing can be done to increase the number of microorganisms, and, therefore, increase the amount of secreted enzymes present. In some embodiments, the sample is not processed. In some embodiments, the sample is not cultured or not placed under conditions that would allow further microorganism growth or an increase in secreted enzyme in the sample.

Samples can be incubated with at least one cellulase substrate that provides a detectable signal upon contact with cellulose or a cellulose substrate. In some cases, a positive signal can be generated, such as going from colorless to colored or non-fluorescent to fluorescent. In other cases, a negative signal can be generated, such as going from colored to colorless or from fluorescent to less fluorescent or non-fluorescent. As an example, a food sample containing a microbial contaminant that secretes a CBH enzyme may be incubated with at least one CBH substrate, for example, a cellobioside derivative having an indicator moiety. The indicator moiety may be a moiety with specific spectroscopic properties such as visible color, UV absorption or fluorescence. The CBH1 enzyme hydrolyzes the cellobioside derivative and releases the indicator moiety. The spectroscopic property of the indicator moiety may be used to determine the presence and the quantity of the microbe contaminant. Samples that do not have microbial contaminants would produce a reduced or zero signal relative to a contaminated sample.

Some embodiments provide methods of detecting the presence of microorganisms (e.g. cellulolytic microorganisms) in a sample. In some embodiments, the method comprises contacting the sample with a secreted enzyme substrate (e.g. cellobiohydrolase substrate). In some embodiments, the substrate comprises an indicator moiety. In some embodiments, the indicator moiety can be used to detect the presence of the secreted enzyme. The indicator moiety can be used as a marker for enzymatic activity because the secreted enzyme will act on the substrate comprising the indicator moiety and releasing the moiety. The release of the moiety can then be detected as evidence and presence of the secreted enzyme. This evidence and activity can then, in some embodiments, be used to detect the presence of microorganisms in the sample. Accordingly, in some embodiments, the method comprises detecting cellobiohydrolase activity. In some embodiments, the detection of cellobiohydrolase activity indicates microorganisms are present in the sample or have contaminated the sample.

In some embodiments, detecting cellobiohydrolase activity comprises detecting release of the indicator moiety from the substrate. In some embodiments, detecting cellobiohydrolase activity comprises detecting hydrolysis of the cellobiohydrolase substrate. In some embodiments, the indicator moiety is not hydrolyzed or released from the substrate by the secreted enzyme. In some embodiments, the indicator moiety and/or the substrate forms a covalent bond or stable interaction with the enzyme and this covalent bond or stable interaction can be detected. In some embodiments, the detection of the covalent bond or stable interaction indicates that bacteria are present in a sample.

In some embodiments, cellobiohydrolase activity may be detected with a spectrometer. The spectrometer may be a UV spectrometer or a florescent spectrometer. Alternatively, cellobiohydrolase activity may be detected visually by eye. The activity can also be detected, for example, using a colorimeter or similar device.

In some embodiments, detecting the release of the indicator moiety includes detecting the release of the indicator moiety with a spectrometer. In some embodiments, the released indicator moiety from the substrate may be UV-active, fluorescent or any combination thereof. In some embodiments, when the indicator moiety or the substrate is not released, but instead forms a covalent bond or stable interaction, the bonding or stable interaction can be detected by a spectrometer. For example, when a substrate with or without an indicator moiety binds to the enzyme, the binding causes a change in the excitation of the light being emitted and this change can be detected using, for example, a spectrometer.

In some embodiments, the indicator moiety is nitrophenol, 4-methylumbelliferone, or any combination thereof. In some embodiments, the indicator moiety is nitrophenol, 4-nitrophenol, 2-chloro-4-nitrophenol, 4-methylumbelliferone, resorufin, 4-methyl-7-thioumbelliferone, or any combination thereof. The specific indicator moiety is not critical so long as the release or non-release event can be detected and the detection of the moiety will indicate the presence of the secreted enzyme, and, therefore, the presence of a microorganism in the sample. Therefore, in some embodiments, the substrate are compounds or compositions that when acted upon by a secreted enzyme release a product, such as the indicator moiety, are UV active or fluorescent compounds. In some embodiments, these products can be quantified, detected, and/or identified using a spectrometer and/or spectrophotometric/fluorescence spectrometric techniques. In some embodiments, the released indicator moiety from the substrate may be, but not limited to, an ultraviolet moiety, a fluorescent moiety or a chromogenic moiety.

In some embodiments, the present invention provides methods of quantifying microorganisms in a sample. In some embodiments, quantifying microorganisms in a sample comprises measuring a rate of release of the indicator moiety from the substrate. Measuring the rate of release can then be used, in some embodiments, to correlate the rate of release of the indicator moiety to an amount of microorganisms present in the sample. In some embodiments, quantifying the microorganisms in the sample comprises calculating area under the curve (AUC) that is generated by detecting cellobiohydrolase activity (e.g. detection and measurement of the indicator moiety).

In some embodiments, the cellobiohydrolase substrate is, but not limited to, 4-methylumbelliferyl-beta-D-cellobioside, 2-nitrophenyl-beta-D-cellobioside, 2-chloro-4-nitrophenyl-beta-D-cellobioside, 4-nitrophenyl-beta-D-cellobioside, cellulose azure, Resorufin cellobioside, 4-Methyl-7-thioumbelliferyl-beta-D-cellobioside, or any combination thereof. Other cellobiohydrolase may also be used.

In some embodiments, the sample comprises microorganisms. In some embodiments, the microorganism is a bacteria. In some embodiments, the microorganism is an aerobic or an anaerobic bacteria. In some embodiments, the microorganism is a fungus, such as a cellulolytic fungus. In some embodiments, the fungus is an anaerobic or aerobic fungus. In some embodiments, the microorganism is, but not limited to, Clostridium, Trichoderma, Cellulomonas, or any combination thereof. In some embodiments, the bacteria is, but not limited to, Clostridium thermocellum and Cellulomonas fimi, or any combination thereof. In some embodiments, the fungus is, but not limited to, Chaetomium, Stachybotrys, Trichoderma (e.g. Trichoderma reesei and Trichoderma viride), or any combination thereof.

In some embodiments, the methods of detecting or quantifying microorganisms in a sample further comprise treating the sample to remove or neutralize the pathogenic properties of the microorganisms. In some embodiments, the sample is treated with an antibiotic to neutralize the bacteria. In some embodiments, the sample is treated with an antifungal to neutralize the fungus. In some embodiments, the sample is irradiated or pasteurized to remove the microorganisms. In some embodiments, if the sample has been taken from a larger composition, the larger composition is treated to remove, eradicate, or neutralize the microorganisms so that ingestion of the composition or coming in contact with the composition will no longer make an individual sick.

Some embodiments provide kits for the detection of microorganisms in a sample. In some embodiments, the kit comprises a cellobiohydrolase substrate. In some embodiments, the substrate comprises an indicator moiety. In some embodiments, the kit comprises instructions for performing a method of detecting microorganisms in a sample. In some embodiments, the kit comprises one or more positive controls. In some embodiments, the kit comprises one or more negative controls. A positive control can be, for example, a sample that is known to contain a specific amount of microorganisms and/or a specific amount of enzyme that can be quantified. In some embodiments, the positive control's enzymatic activity has been correlated with a specific amount of microorganisms in a sample. A negative control can be, in some embodiments, a sample that does not comprise any microorganisms or may contain microorganisms but does not have active secreted enzyme. The negative control may have a protein that is a secreted enzyme but the secreted enzyme can be inactivated, by for example, heat denaturation, chemical denaturation, and the like. The instructions included in the kit, in some embodiments, provide direction for performing a method of detecting microorganisms in a sample. In some embodiments, the instructions provide directions that include, but are not limited to, contacting a sample with a cellobiohydrolase substrate and detecting cellobiohydrolase. The instructions can also explain and provide direction for the detection of a substrate comprising an indicator moiety. The instructions can also provide direction for how the detection of the enzymatic activity indicates that microorganisms are present in the sample. The instructions, in some embodiments, can also provide directions for quantifying an amount of microorganisms present in a sample.

The kit can also, in some embodiments, comprise a substrate comprising an indicator moiety as described herein. The substrate can also be any substrate including, but not limited to, the substrates described herein. For example, the substrate comprising the indicator moiety can be, but is not limited to, 4-methylumbelliferyl-beta-D-cellobioside, 2-nitrophenyl-beta-D-cellobioside, 2-chloro-4-nitrophenyl-beta-D-cellobioside, 4-nitrophenyl-beta-D-cellobioside, cellulose azure, Resorufin cellobioside, 4-Methyl-7-thioumbelliferyl-beta-D-cellobioside, or any combination thereof.

In some embodiments, the kit is configured for detection of anaerobic bacteria. In some embodiments, the kit is configured for detection of Clostridium, Trichoderma, Cellulomonas, or any combination thereof. In some embodiments, the kit is configured for detection of Clostridium thermocellum, Trichoderma reesei, Cellulomonas fimi, or any combination thereof “Configured for detection” refers to a kit that specifically includes reagents, protocols, components, and/or compositions for the detection of a specific microorganisms, including but not limited to cellulolytic bacteria or fungus and those described herein. For example, in some embodiments, a kit that is configured to detect a first microorganism, such as Clostridium, can have reagents that are specific for the first bacteria, whereas a kit that is configured to detect a second microorganism, such as Trichoderma, can have different reagents than those present in the kit configured to detect the first microorganism. In some embodiments, a kit that is configured for the detection of a first microorganism comprises the same reagents as a kit that is configured for detection of a second microorganism. In some embodiments, kits configured for the detection of different microorganisms may differ only by the difference in protocol or instructions. In some embodiments, kits configured for detection of different microorganisms comprises different substrates.

A kit can also be configured to detect different types of microorganisms. In some embodiments, a kit is configured to detect at least 2, 3, 4, or 5 different types of microorganisms. In some embodiments, the kit comprises at least two different substrates to detect at least two different microorganisms. In some embodiments, the kit is configured to detect the different microorganisms simultaneously or separately (i.e. sequentially). In some embodiments, a substrate that is specific to a microorganism enzyme other than a CBH is in the kit. Accordingly, in some embodiments, the two different substrates can be used to detect different microorganisms in the same sample.

Some embodiments provide a method of preparing a kit for the detection of microorganisms in a sample. In some embodiments, the method of preparing a kit comprises placing in a container a cellobiohydrolase substrate comprising an indicator moiety. In some embodiments, the method comprises placing instructions for performing a method of detecting microorganisms in a sample, the method comprising contacting the sample with a cellobiohydrolase substrate, the substrate comprising an indicator moiety; and detecting cellobiohydrolase activity, wherein detection of cellobiohydrolase-1 activity indicates a microorganism is present in the sample. In some embodiments, the method of preparing a kit comprises placing one or more of a positive control in the kit. In some embodiments, the method comprises placing a negative control in the kit. In some embodiments, the kit comprises a container that can contain the items present in the kit.

Some embodiments provide compositions. In some embodiments, the composition comprises a sample. In some embodiments, the composition comprises a secreted enzyme, such as but not limited to, a cellobiohydrolase. In some embodiments, the composition comprises a cellobiohydrolase substrate. In some embodiments, the composition comprises a cellobiohydrolase substrate comprising an indicator moiety. In some embodiments, the composition comprises any combination of any sample, any enzyme, any substrate, and any indicator moiety described herein. For example, in some embodiments, the composition comprises a sample, a cellobiohydrolase, a cellobiohydrolase substrate with or without an indicator moiety. In some embodiments, the sample has been processed or purified as described herein or otherwise processed. In some embodiments, the sample is a food sample. In some embodiments, the cellobiohydrolase substrate comprising an indicator moiety is, but not limited to, 4-methylumbelliferyl-beta-D-cellobioside, 2-nitrophenyl-beta-D-cellobioside, 2-chloro-4-nitrophenyl-beta-D-cellobioside, 4-nitrophenyl-beta-D-cellobioside, cellulose azure, Resorufin cellobioside, 4-Methyl-7-thioumbelliferyl-beta-D-cellobioside, or any combination thereof. In some embodiments, the composition comprises a substrate that has been acted upon by a cellobiohydrolase. In some embodiments, the composition comprises a hydrolyzed cellobiohydrolase substrate.

### Example 1

### Use of p-Nitrophenyl β-D-Cellobioside for Detection of Bacterial Contamination in Food

P-nitrophenyl β-D-cellobioside, which contains a chromogenic moiety, can be added as a substrate for CBH-1 to a sample of milk suspected of being contaminated with bacteria. The combination of the milk and the substrate can be placed at 37° C. for one hour. CBH-1 producing bacteria will hydrolyze the substrate (e.g., p-nitrophenyl β-D-cellobioside) to release p-nitrophenol. After the incubation period, the composition can be placed in a spectrometer to detect fluorescence at 366 nm. If fluorescence UV activity is detected, it will indicate the presence of bacteria in the milk sample. The detection will also indicate that the sample is contaminated with bacteria. The rate of hydrolysis or the hydrolytic activity in a sample will be proportional to the amount of florescence released and the amount of bacteria is quantified. Fluorescence from release of p-nitrophenol will not be detected in a negative sample of uncontaminated milk.

### Example 2

### Determination of the Presence or Absence of a Bacteria in Sample

4-methylumbelliferyl-beta-D-cellobioside, which contains a chromogenic moiety, can be added as a substrate for CBH-1 to a soil sample. The soil sample can be mixed in a solvent, such as water, and incubated with the substrate at 37° C. for one hour. The sample can be analyzed for the release of 4-methylumbelliferone. The analysis can be performed in a spectrometer to detect fluorescence of any indicator moiety that has been released. The spectrometer will fail to detect any fluorescence in excess of what is observed with the negative control. The result will indicate that sample is not contaminated with bacteria.

### Example 3

### Method of Quantifying Bacteria in a Food Sample

Drinking water can be incubated with a CBH-1 substrate that contains a UV-indicator moiety. The UV absorbance can be measured with a spectrometer. A curve can be calculated based upon the UV absorbance and the amount of bacteria in the drinking water can be determined by calculating the area under the curve (AUC). The amount of the bacteria thus is determined will be found to be unsafe and the drinking water can optionally be treated or purified to remove the contamination.

### Example 4

### Method of Detecting Clostridium thermocellum

A poultry food sample can be taken and mixed with an aqueous solvent. The mixture can be cultured, if necessary, to allow any bacteria present in the sample to grow. A kit configured for the detection of Clostridium thermocellum can be used to process the sample. The kit will contain a positive control and a negative control against which the sample's results are compared. The kit can also contain an instruction manual for the detection of Clostridium thermocellum. The kit can contain a substrate that is specific for CBH-1. The substrate can be incubated with the mixture containing the poultry food sample at 37° C. and the sample can be analyzed for the release of the indicator moiety. The indicator moiety can be detected with a spectrometer and the sample can be found to contain Clostridium thermocellum. The sample can be further analyzed to quantify the amount of Clostridium thermocellum present in the sample by determining the AUC. The sample can be found to have a significant amount of Clostridium thermocellum in the sample.

### Example 5

### Method of Detecting T. reesei

A meat food sample can be taken and mixed with an aqueous solvent. The mixture can be cultured, if necessary, to allow any fungus present in the sample to grow. A kit configured for the detection of T. reesei can be used to process the sample. The kit can contain a positive control and a negative control against which the sample's results are compared. The kit can also contain an instruction manual for the detection of T. reesei. The kit can contain a substrate that is specific for CBH-1. The substrate can be incubated with the mixture containing the poultry food sample at 37° C. and the sample can be analyzed for the release of the indicator moiety. The indicator moiety can be detected with a spectrometer and the sample can be found to contain T. reesei. The sample can be further analyzed to quantify the amount of T. reesei present in the sample by determining the AUC. The sample can be found to have a significant amount of T. reesei in the sample.

### Example 6

### Detection of Trichoderma reesei and Cellulomonas fimi in Food

A sample suspected of being contaminated with a microorganism can be analyzed with a kit that is configured to detect both Trichoderma reesei and Cellulomonas fimi. The kit can contain enzymatic substrates that are specific for Trichoderma reesei or Cellulomonas fimi. The pork food sample can be mixed with a solvent and then incubated with the substrate that is specific for Trichoderma reesei. The sample can be analyzed for the release of the indicator moiety that is present in the substrate that is specific for Trichoderma reesei. Trichoderma reesei can be detected based upon the results obtained. The sample can then be analyzed for the presence of Cellulomonas fimi. The sample can be further incubated with the substrate that is specific for Cellulomonas fimi. The sample can be analyzed for the release of the indicator moiety that is released when the substrate that is specific for Cellulomonas fimi is released. The spectrometer can detect the release of the moiety. The sample can be found to contain Cellulomonas fimi.

### Example 7

### Preparation of Kit Configured for the Detection of Bacteria

A kit for the detection of Clostridium, Trichoderma, or Cellulomonas can be assembled. The kit can be configured as a container (such as a box). A substrate that can be cleaved by a secreted enzyme (CBH-1) can be added to the container. A negative control and a positive control can also be added to the container. An instructional manual can be added to the container. The kit can be sealed and prepared for shipment.

### Example 8

### Detection of Bacteria from on a Counter

A countertop can be swabbed. The swab can be mixed with bacteria culture media. The culture media can be incubated at 37° C. for about 4 hours. The culture can then be incubated with any CBH-1 substrate with an indicator moiety (4-methylumbelliferyl-beta-D-cellobioside, 2-nitrophenyl-beta-D-cellobioside, 2-chloro-4-nitrophenyl-beta-D-cellobioside, 4-nitrophenyl-beta-D-cellobioside, cellulose azure, Resorufin cellobioside, or 4-Methyl-7-thioumbelliferyl-beta-D-cellobioside) The indicator moiety can be released by the enzyme and the indicator moiety can be detected by a spectrometer. The countertop can be found to be contaminated with bacteria. The bacteria can also be quantified by determining the AUC and correlating the AUC with the amount of bacteria present on the countertop.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods, kits, compositions, and apparatuses within the scope of the disclosure, in addition to those enumerated herein, will be apparent to those skilled in the art from the foregoing descriptions. Such modifications and variations are intended to fall within the scope of the appended claims. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” and “comprising” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include, but not be limited to, systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method of detecting cellulolytic microorganism contamination of a sample, the method comprising:
contacting the sample with a cellobiohydrolase-1 substrate, the substrate comprising an indicator moiety; and
detecting cellobiohydrolase-1 activity, wherein detection of cellobiohydrolase-1 activity indicates cellulolytic microorganism contamination of the sample.

2. The method of claim 1, wherein the cellulolytic microorganism is a bacteria or a fungus.

3. The method of claim 1, wherein detecting cellobiohydrolase-1 activity comprises detecting release of the indicator moiety.

4. The method of claim 1, wherein the sample is a food sample.

5. The method of claim 1, wherein the sample is suspected of being contaminated with a cellulolytic microorganism.

6. The method of claim 1, wherein detecting cellobiohydrolase-1 activity comprises detecting hydrolysis of the cellobiohydrolase-1 substrate.

7. The method of claim 1, wherein detecting cellobiohydrolase-1 activity comprises detecting the cellobiohydrolase-1 activity with a spectrometer.

8. The method of claim 7, wherein the spectrometer is a UV spectrometer or a fluorescent spectrometer.

9. The method of claim 3, wherein detecting the release of the indicator moiety comprises detecting the release of the indicator moiety with a spectrometer.

10. The method of claim 1, the method further comprising quantifying the cellulolytic microorganism in the sample.

11. The method of claim 10, wherein quantifying comprises measuring a rate of release of the indicator moiety and correlating the rate of release of the indicator moiety to an amount of cellulolytic microorganism present in the sample.

12. The method of claim 10, wherein quantifying the cellulolytic microorganism in the sample comprises calculating area under the curve (AUC) that is generated by detecting cellobiohydrolase-1 activity.

13. The method of claim 1, wherein the cellobiohydrolase-1 substrate is 4-methylumbelliferyl-beta-D-cellobioside, 2-nitrophenyl-beta-D-cellobioside, 2-chloro-4-nitrophenyl-beta-D-cellobioside, 4-nitrophenyl-beta-D-cellobioside, cellulose azure, Resorufin cellobioside, 4-Methyl-7-thioumbelliferyl-beta-D-cellobioside or any combination thereof.

14. The method of claim 2, wherein the bacteria is an aerobic, an anaerobic bacteria, or a combination thereof.

15. The method of claim 1, wherein the cellulolytic microorganism is Clostridium, Trichoderma, Cellulomonas, or any combination thereof.

16. The method of claim 1, wherein the cellulolytic microorganism is Clostridium thermocellum, Trichoderma reesei, Cellulomonas fimi, or any combination thereof.

17. The method of claim 3, wherein released indicator moiety from the substrate is UV-active, fluorescent or any combination thereof.

18. The method of claim 3, wherein the indicator moiety comprises nitrophenol, 4-methylumbelliferone, or any combination thereof.

19. The method of claim 1, wherein the indicator moiety comprises 2-nitrophenol, 4-nitrophenol, 2-chloro-4-nitrophenol, 4-methylumbelliferone, resorufin, 4-methyl-7-thioumbelliferone or any combination thereof.

20. A kit for detection of cellulolytic microorganism contamination in a sample, the kit comprising:
a cellobiohydrolase-1 substrate comprising an indicator moiety;
instructions for performing a method of detecting bacterial or fungal contamination in a sample, the method comprising contacting the sample with a cellobiohydrolase-1 substrate, the substrate comprising an indicator moiety; and detecting cellobiohydrolase-1 activity, wherein detection of cellobiohydrolase-1 activity indicates cellulolytic microorganism contamination is present in the sample; and
optionally, one or more of: a positive control; and a negative control.

21-28. (canceled)

29. A method of preparing the kit of claim 20 comprising placing in a container:
a cellobiohydrolase-1 substrate comprising an indicator moiety;
instructions for performing a method of detecting cellulolytic microorganism contamination in a sample, the method comprising contacting the sample with a cellobiohydrolase-1 substrate, the substrate comprising an indicator moiety; and
detecting cellobiohydrolase-1 activity, wherein detection of cellobiohydrolase-1 activity indicates cellulolytic microorganism contamination is present in the sample;
optionally one or more of a positive control; and
optionally a negative control.

30. A composition comprising a sample, cellobiohydrolase-1, and a cellobiohydrolase-1 substrate comprising an indicator moiety.

31-33. (canceled)
